(19) United States
(12) Patent Application Publication
(10) Pub. No.: US 2021/0361962 A1
(43) Pub. Date: Nov. 25, 2021

(54) DEVICES AND METHODS FOR CONTACTLESS DELIVERING PULSED ELECTRIC FIELDS TO SKIN TISSUE

(71) Applicants: DAHSHIARN CHIAO, Naperville, IL (US); CHIEN-AN CHANG, Taoyuan (TW)

(72) Inventors: DAHSHIARN CHIAO, Naperville, IL (US); CHIEN-AN CHANG, Taoyuan (TW)

(21) Appl. No.: 15/929,767

(22) Filed: May 21, 2020

(51) Int. Cl. A61N 1/44; A61N 1/04

(52) U.S. Cl. CPC A61N 1/44; A61N 1/0448

(57) ABSTRACT

Devices and methods for contactless delivery to a tissue of a subject using applied pulsed electric fields. The methods for controlling a therapy provided to a skin tissue of a subject using applied pulsed electric fields. The methods includes an jetting-nozzle assembly configured to not in directly contact with a skin tissue of a subject to deliver a series of electric field pulses to the skin tissue and a user input configured to receive an operational instruction for the series of electric field pulses. The operational instruction defines at least one of a pulse duration, a pulse frequency, a pulse number, and a pulse amplitude. The methods also includes at least one processor configured to access the operational instruction received by the user input and, using the operational instruction, create an electric field profile to be generated by the jetting-nozzle assembly about the skin tissue of the subject to control a cosmetic skin treatment.

### FIELD OF THE INVENTION

The present invention provides a devices and methods for contactless delivering pulsed electric fields in a manner that controls or promotes skin tissue repair, enhancement, and disinfection. In particular, devices and methods provided may use non-thermal pulsed electric fields delivered periodically or intermittently to control a skin cosmetic treatment.

### BACKGROUND OF THE INVENTION

The present disclosure relates generally to devices and methods for contactless delivering pulsed electric fields to skin tissue and, in particular, to devices and methods for controlling and cosmetic treating skin.

An alternative approach to skin cosmetic treatment is the implementation of electrical stimulation. The rationale for using electrical stimulation is based on the fact that the human body has endogenous bioelectric systems that promote wound and skin healing. However, when the body's endogenous bioelectric system is inadequate, external electrical stimulation can be used to supplement the natural bioelectric currents or electric fields for enabling or enhancing wound healing and skin cosmetic treatment.

There are several disadvantages associated with prior art methods of electrical stimulation for wound healing and skin cosmetic treatment. One disadvantage is that many prior art methods require placement of one or perhaps two electrodes directly on the skin tissue. Such placement increases the probability of bacterial contamination, thereby complicating wound healing and skin cosmetic treatment. Other prior art devices and methods are inconvenient or difficult to employ as a result of their bulk or complexity. For example, several prior art devices require the implementation of several electrodes, whereby one electrode is applied directly over the skin area or a pulsed electromagnetic field treatment coil is placed directly over the skin area for inducing pulsed electric fields to the skin. This makes extended treatment periods uncomfortable for the patient, as well as prohibiting free travel of the patient.

Thus, it is desirable to provide an improved electrical stimulation method for promoting wound healing and skin cosmetic treatment. The method should treat the wound area without actual contact with the wound to reduce the probability of bacterial infection. Further, the method should be simple and inexpensive while effectively treating wounds and non-wounded skin tissue.

### SUMMARY OF THE INVENTION

The limitations of the prior art are overcome in methods of the present invention by providing devices and methods for contactless delivering pulsed electric fields in a manner that controls or promotes skin tissue repair, enhancement, and disinfection. In particular, devices and methods provided may use non-thermal pulsed electric fields delivered periodically or intermittently to control a skin cosmetic treatment. A first method of the present invention comprises a specific structure for a jetting-nozzles configuration to deliver a series of pulsed ion stream and electrical gradient to the skin tissue. The specific structure comprises a physical configuration mounted in fixed relation to the body of the device and a ring configuration is in the form of an annular cable mounted on the body of device, and usually in substantially concentric relation with the jetting-nozzles unit. The ring configuration and the jetting-nozzles unit may however be adjustable with respect to one another in the direction of jetting. Where the jetting-nozzles and/or the ring configuration is adjustable, preferably the limits of adjustment are such that the jetting-nozzles configuration, over substantially its full range of adjustment, has its forward extremity located forwardly of the jetting-nozzles. The arrangement is conveniently such that, in all positions of relative adjustment, jetting is focused when the forward extremity of the jetting-nozzles configuration is within a certain distance from an earthed skin tissue target. In addition, the ring configuration surrounds the jetting-nozzles and yields the necessary electric field around the jetting-nozzles during the application of pulsed electric fields.

A second method of the present invention comprises a specific structure or design for a grounding configuration to assist in generating electrical gradient to the skin tissue. The specific structure comprises a physical configuration mounted in fixed relation to the jetting-nozzles unit, a grounding ring configuration is in the form of an annular cable mounted on the body of device, and an external grounding wire configuration is directly connected with the skin tissue. The arrangement is conveniently such that, pulsed ion stream jetting is focused when the forward extremity of the jetting-nozzles configuration is within a certain distance from a grounding skin tissue target. In addition, the grounding ring configuration surrounds the jetting-nozzles and yields the necessary electrical gradient between the jetting-nozzles and a skin tissue during the application of pulsed electric fields.

A third method of the present invention comprises a reservoirs method. Each reservoir further comprises a high voltage charged administrable material storage region. The material storage region comprises a material storage configuration. In general where the stored material to be jetted is in the form of a liquid, a gas or a powder, the high voltage input will have the effect of causing the propulsion of one or more filaments or ligaments of stored material from the jetting-nozzle, which ligament(s) break up into positive or negative charged droplets for transporting ion stream to the targeted skin tissue and also generating an electrical gradient with the targeted grounding skin tissue.

A fourth method of the present invention comprises a hand-held pulsed electric fields device which having at least one reservoir, at least one jetting-nozzles configuration, a ring configuration, a grounding ring configuration, a high voltage generator, a power source, an external grounding wire configuration, a controller, a grounding circuit design, a trigger. The special arranged grounding configuration is directly connected the skin tissue with an external grounding wire configuration through the grounding circuit design. Through the special arranged grounding configuration the targeted human skin will be earthed properly.

In yet another variant of the fourth method of the present invention includes an jetting-nozzles assembly configured to engage a skin tissue of a subject to contactless deliver a series of electric field pulses to the skin tissue and a user input configured to receive an operational instruction for the series of electric field pulses, the operational instruction defining at least one of a pulse duration, a pulse frequency, a pulse number, and a pulse amplitude. The system also includes at least one processor configured to access the operational instruction received by the user input and using the operational instruction, create an electric field profile to be generated by the jetting-nozzles assembly about the skin tissue of the subject to control a skin cosmetic treatment.

### BRIEF DESCRIPTION OF THE FIGURES

The above and other objects and advantages of this invention will be apparent upon consideration of the following detailed description, taken in conjunction with the accompanying drawings in which like characters refer to like elements throughout and in which:

FIG. 1 depicts front and side views of a hand-held pulsed electric fields device having a jetting-nozzles configuration, a ring configuration, a grounding ring configuration, and an external grounding wire configuration made in accordance with a preferred embodiment of the present invention.

FIG. 2 depicts side and perspective views of a hand-held pulsed electric fields device having a jetting-nozzles configuration, a ring configuration, a grounding ring configuration, an external grounding wire configuration, a grounding circuit design, a high voltage generator, a controller, a power source, and reservoirs made in accordance with a preferred embodiment of the present invention.

FIG. 3 depicts rear and perspective views of a hand-held pulsed electric fields device having a jetting-nozzles configuration, a ring configuration, a grounding ring configuration, an external grounding wire configuration, a grounding circuit design, a high voltage generator, a controller, a power source, and reservoirs made in accordance with a preferred embodiment of the present invention.

FIG. 4 is a flowchart illustrating the steps associated with a mode of operation of a method, in accordance with the present invention.

### DETAILED DESCRIPTION OF THE PREFERRED EMBODIMENTS

For the purpose of promoting an understanding of the principles of the invention, reference will now be made to the embodiments illustrated in the drawings and specific language will be used to describe the same. It will nevertheless be understood that no limitation of the scope of the invention is thereby intended, such alterations and further modifications in the illustrated device and such further applications of the principles of the invention as illustrated therein being contemplated as would normally occur to one skilled in the art to which the invention relates.

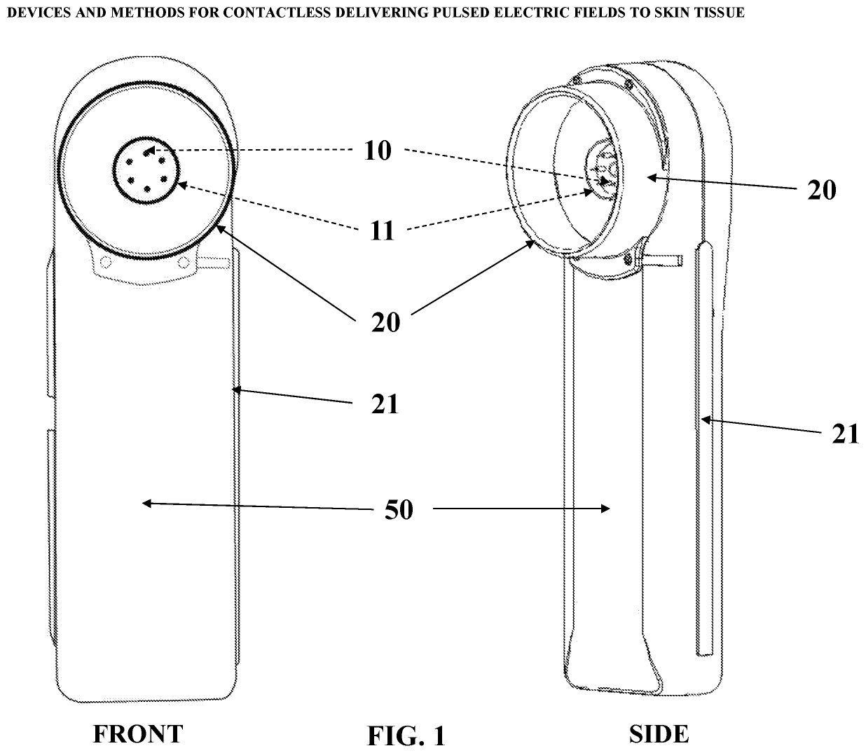

A hand-held pulsed electric fields device 50 made in accordance with the first and second preferred embodiments of the present invention is depicted in front and side views in FIG. 1. The jetting-nozzles configuration 10 mounted in fixed relation to the body of the device 50 and a ring configuration 11 is in the form of an annular cable mounted on the body of device 50, and usually in substantially concentric relation with the jetting-nozzles unit 10. The ring configuration 11 and the jetting-nozzles unit 10 may however be adjustable with respect to one another in the direction of jetting.

The grounding ring configuration 20 mounted in fixed relation to the jetting-nozzles unit 10 in the form of an annular cable mounted on the body of device 50, and an external grounding wire configuration 21 is directly connected with the skin tissue. The grounding ring configuration 20 surrounds the jetting-nozzles 10 and yields the necessary electrical gradient between the jetting-nozzles 10 and skin tissue during the application of pulsed electric fields.

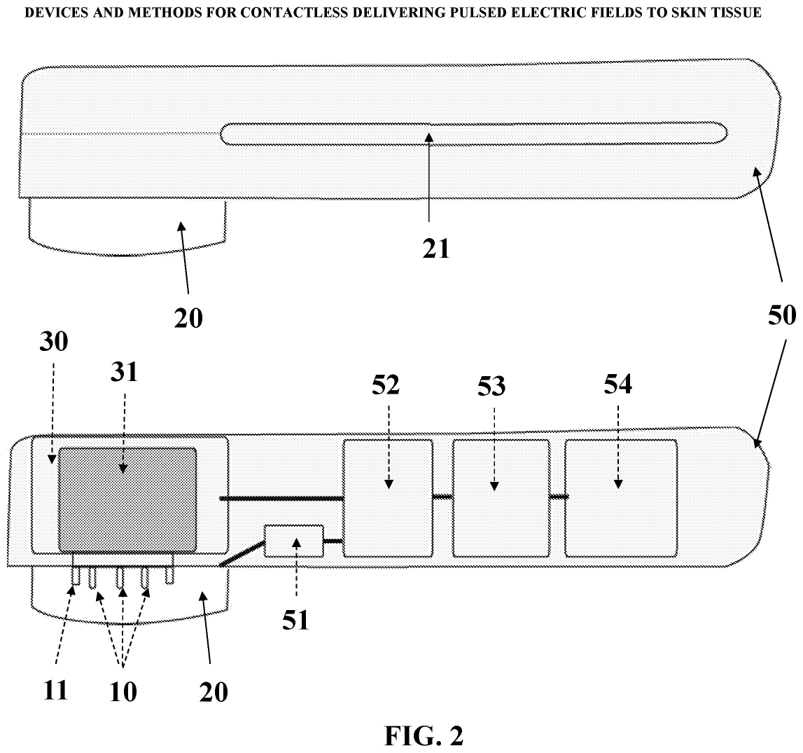

A reservoir made in accordance with the third preferred embodiments of the present invention is depicted in side and perspective views in FIG. 2. The specific method comprises a reservoirs configuration 30. Each reservoir further comprises a high voltage charged administrable transporting material storage region 31. The transporting material storage region 31 comprises a material storage configuration. In general the high voltage input 52 will have the effect of causing the propulsion of one or more filaments or ligaments of stored material from the jetting-nozzle 10, which ligament(s) break up into positive or negative charged droplets for transporting ion stream to the targeted skin tissue and also generating an electrical gradient with the grounding skin tissue.

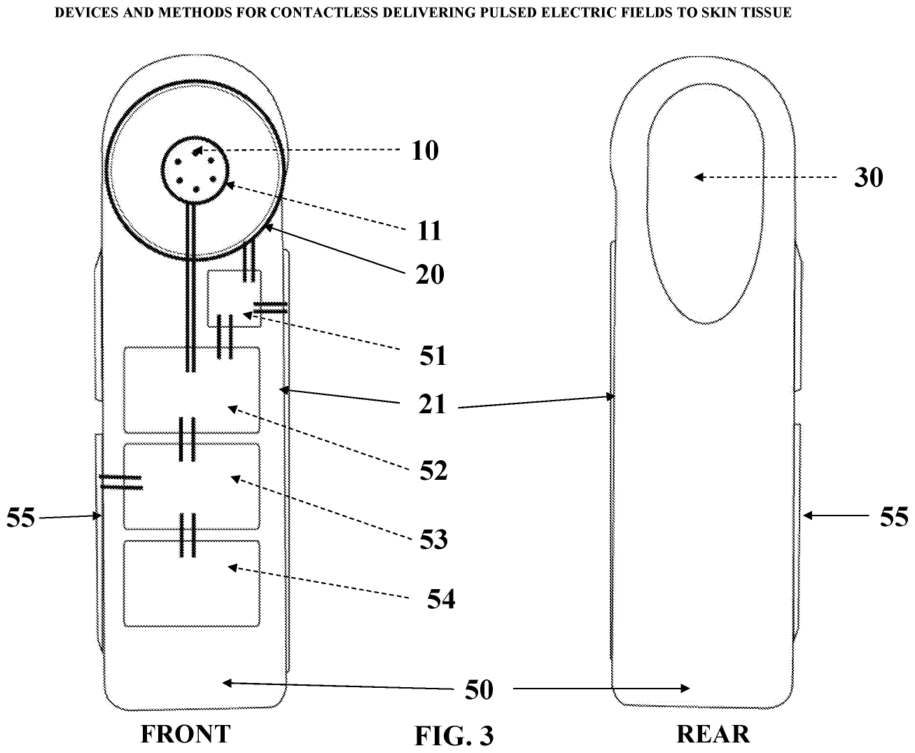

A hand-held pulsed electric fields device 50 made in accordance with the fourth preferred embodiments of the present invention is depicted in rear and perspective views in FIG. 3. hand-held pulsed electric fields device 50 generally have at least one reservoir 30, at least one jetting-nozzles configuration 10, a ring configuration 11, a grounding ring configuration 20, a high voltage generator 52, a power source 54, an external grounding wire configuration 21, a controller 53, a grounding circuit design 51, a trigger 55. The special arranged grounding configuration is directly connected the skin tissue with an external grounding wire configuration 21 through the grounding circuit design 51. Through the special arranged grounding configuration the targeted human skin will be earthed properly.

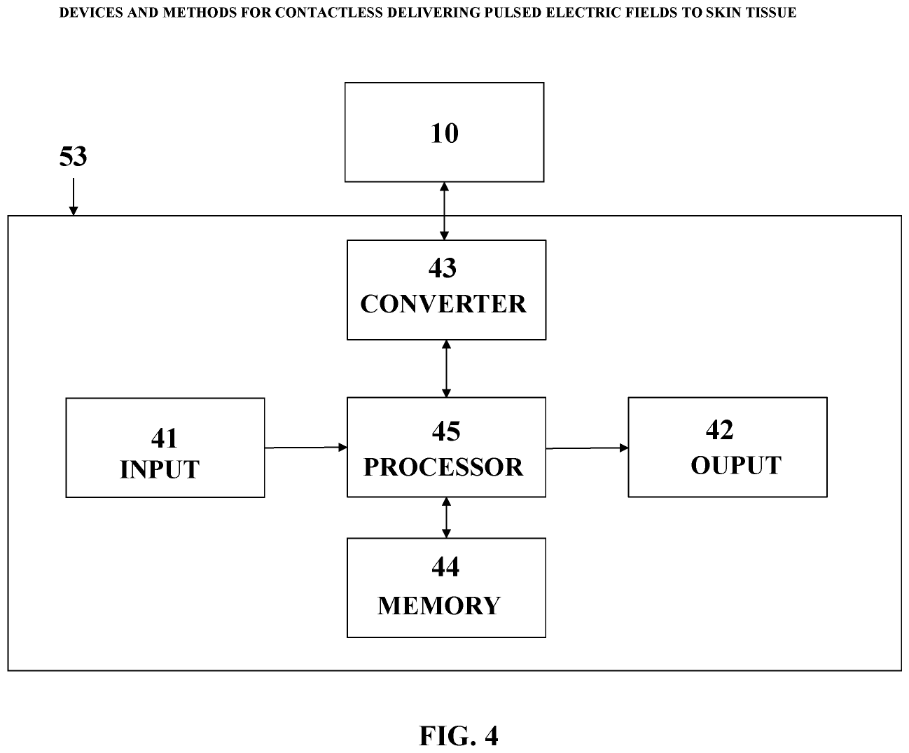

A hand-held pulsed electric fields device 50 made in accordance with the fourth preferred embodiments of the present invention is depicted in a plan view in FIG. 4. The hand-held pulsed electric fields device 50 generally includes a controller 53, configured to generate, transmit and receive electronic signals from a jetting-nozzles configuration 10 generally not in contacting a subject, and may also include a multi-channel converter 43 configured to convert digital to analog and analog to digital signals. An operational instruction defining the electric field pulses delivered by the controller 53 to the subject via the jetting-nozzles configuration 10 may be provided by an operator, using any number of controllable input parameters, that may include pulse duration, pulse frequency, pulse number, pulse amplitude. Typical ranges of numerical values describing the electric field pulses may include 50 nanoseconds to 500 millisecond for the pulse durations, 0.05 to 10000 Hertz for the pulse frequencies, 1 to 10000 for the pulse numbers, and 1 to 250000 Volts for the pulse amplitudes, although other values may be possible.

The invention claimed is:

1. A method for the contactless delivery of pulsed electric fields to a skin tissue, comprising: contactless delivery to the skin tissue with a jetting-nozzles configuration to deliver a series of pulsed ion stream and electrical gradient to a grounding skin tissue. The specific structure comprises a physical configuration mounted in fixed relation to the body of the device and a ring configuration is in the form of an annular cable mounted on the body of device, and usually in substantially concentric relation with the jetting-nozzles unit. The ring configuration and the jetting-nozzles unit are connected with a high voltage input, and may however be adjustable with respect to one another in the direction of ion stream jetting. And a special arranged grounding configuration from the body of the device is directly connected the skin tissue with an external grounding wire configuration through a grounding circuit design.

2. The method according to claim 1, wherein the jetting-nozzles and/or the ring configuration is adjustable and connected with a high voltage input, preferably the limits of adjustment are such that the nozzle-ring configuration, over substantially its full range of adjustment, has its forward extremity located forwardly of the jetting-nozzles to deliver a series of pulsed ion stream and electrical gradient to a grounding skin tissue.

3. The method according to claim 1, wherein the jetting-nozzles and/or the ring configuration is adjustable and connected with a high voltage input, preferably the limits of adjustment are such that the nozzle-ring configuration, over substantially its full range of adjustment, has its forward extremity located forwardly of the jetting-nozzles to deliver ion stream and electrical gradient to a grounding skin tissue.

4. The method according to claim 1, wherein the ring configuration surrounds the jetting-nozzles and yields the necessary electric field around the jetting-nozzles during the application of electric fields to the skin tissue.

5. The method according to claim 1, wherein the stream to be delivered is in the form of a ion or an electron will have the effect of causing electric fields to a grounding skin tissue.

6. A method for the contactless delivery of pulsed electric fields to a skin tissue, comprising: contactless delivery a series of pulsed ion stream and electrical gradient to a grounding skin tissue with a specific structure for a grounding configuration to generate electrical gradient to a skin tissue. The specific structure comprises a physical configuration mounted in fixed relation to the jetting-nozzles unit which jetting pulsed ion stream, a grounding ring configuration is in the form of an annular cable mounted on the body of device, and an external grounding wire configuration is directly connected with the skin tissue. The grounding ring configuration surrounds the jetting-nozzles and yields the necessary electrical gradient between the jetting-nozzles and a grounding skin tissue during the application of pulsed electric fields.

7. The method according to claim 2, wherein the pulsed ion stream jetting is focused when the forward extremity of the jetting-nozzles configuration is within a certain distance from a grounding skin tissue target, and the jetting-nozzles configuration is not in direct contact with the grounding skin tissue.

8. A method for the contactless delivery of pulsed electric fields to a skin tissue, comprising: contactless delivery with transporting particles to deliver a series of pulsed ion stream and electrical gradient to a grounding skin tissue. The specific method comprises a reservoirs configuration. Each reservoir further comprises a high voltage charged administrable transporting material storage region. The transporting material storage region comprises a material storage configuration. In general the high voltage input will have the effect of causing the propulsion of one or more filaments or ligaments of stored material from the jetting-nozzle, which ligament(s) break up into positive or negative charged droplets for transporting ion stream to the targeted skin tissue and also generating an electrical gradient with the grounding skin tissue.

9. The method according to claim 3, wherein the stored material to be jetted is in the form of a liquid, a gas, a powder or a nanoparticle, the high voltage input will have the effect of causing the propulsion of one or more filaments or ligaments of stored material from the jetting-nozzle.

10. The method according to claim 3, wherein a contactless delivery of pulsed ion stream and electrical gradient to the skin tissue with or without transporting particles.

11. The method according to claim 3, wherein the reservoirs for storing at least one bioactive agent.

12. The method according to claim 3, wherein a charged bioactive particle applying to the tissue a pulsed electric fields, such that the charged bioactive transport through transiently permeabilized tissue by electroporation.

13. The method according to claim 3, wherein the reservoirs configuration comprises at least one replaceable reservoir.

14. A hand-held device for the contactless delivery of pulsed electric fields to a skin tissue, comprising: contactless delivery a series of pulsed ion stream and electrical gradient to a grounding skin tissue with at least one reservoirs configuration, at least one jetting-nozzles configuration, a ring configuration, a grounding ring configuration, a high voltage generator, a power source, an external grounding wire configuration, a controller, a grounding circuit design, a trigger. The special arranged grounding configuration is directly connected the skin tissue with an external grounding wire configuration through the grounding circuit design. Through the special arranged grounding configuration the targeted human skin will be earthed properly.

15. The method according to claim 4, wherein the controller includes a user input configuration to receive an operational instruction for the series of electric field pulses, the operational instruction defining at least one of a pulse duration, a pulse frequency, a pulse number, and a pulse amplitude.

16. The method according to claim 4, wherein the controller also includes at least one processor configured to access the operational instruction received by the user input and using the operational instruction, create an electric field profile to be generated by the jetting-nozzles assembly about the skin tissue of the subject to control a skin cosmetic treatment.

17. The method according to claim 4, wherein the controller generates the electric field parameters includes selecting at least one of: a range between 50 nanoseconds and 500 milliseconds for the pulse duration, a range between 0.05 and 10000 Hertz for the pulse frequency, a range between 1 and 10000 pulses for the pulse number, and a range between land 25000 Volts for the pulse amplitude.

18. The method according to claim 4 and further comprising delivering the pulsed electric field, based on the electric field parameters, to the skin tissue a plurality of times with pauses between each delivery.

19. The method according to claim 4, wherein the jetting-nozzles and/or the ring configuration is connected with a high voltage generator, has its forward extremity located forwardly of the jetting-nozzles to deliver ion stream and electrical gradient to a grounding skin tissue.

20. The method according to claim 4, wherein the reservoirs configuration comprises at least one replaceable reservoir.
